(19) United States
(12) Patent Application Publication
(10) Pub. No.: US 2013/0089907 A1
(43) Pub. Date: Apr. 11, 2013

(54) HYDROGEN PRODUCTION METHOD USING ALCOHOL AND PHOTOSYNTHETIC BACTERIA

(71) Applicants: Mi-Sun Kim, Daejeon (KR); Jeong-Kug Lee, Seoul (KR); Eui-Jin Kim, Seoul (KR); Dong-Hoon Kim, Gwangju (KR); Hyae-Jeong Hwang, Seoul (KR)

(72) Inventors: Mi-Sun Kim, Daejeon (KR); Jeong-Kug Lee, Seoul (KR); Eui-Jin Kim, Seoul (KR); Dong-Hoon Kim, Gwangju (KR); Hyae-Jeong Hwang, Seoul (KR)

(21) Appl. No.: 13/702,237

(22) PCT Filed: Oct. 14, 2011
(86) PCT No.: PCT/KR11/07660

§ 371 Date: Dec. 5, 2012

(30) Foreign Application Priority Data
Oct. 15, 2010 (KR) 10-2010-0100637

(51) Int. Cl. C12P 3/00

(52) U.S. Cl. CPC C12P 3/00
USPC 435/168

(57) ABSTRACT

The present disclosure relates to methods for producing hydrogen using photosynthetic bacteria comprising a step of culturing the photosynthetic bacteria in the presence of alcohol at the condition under which the photosynthesis occurs. The present methods are cost-effective and have a high applicability due to the increased hydrogen productivity compared to the conventional methods in addition to not being sensitive by the inhibitory action by ammonium ion present in the culture. Thus the present methods are particularly useful for producing hydrogen using organic wastes which contains large amount of ammonia therein.

### CROSS-REFERENCE TO RELATED APPLICATIONS

This application is a National Stage of PCT international Patent Application No. PCT/KR2011/007660, filed Oct. 14, 2011, and claims the benefit of Korean Patent Application No. 2010-0100637, filed Oct. 15, 2010, in the Korean Intellectual Property Office, the disclosure of which are incorporated herein by reference.

### BACKGROUND OF INVENTION

1. Field of the Invention

The present disclosure relates to methods for producing hydrogen using photosynthetic bacteria, particularly, to methods for producing hydrogen using photosynthetic bacteria comprising culturing the bacteria in a medium containing alcohol, or to methods for improving the efficiency of hydrogen production.

2. Description of the Related Art

The modern industries have been built on a system that heavily depends on fossil fuels to power the industry and manufacturing, and to provide the electricity. However, the supplies of fossil fuels are limited and eventually the degree to which we depend on fossil fuels should be lessened as the continued use of fossil fuels will cause economical as well as environmental problems. Therefore in recent years, much effort has been devoted for developing the alternative power sources, such as solar energy to replace the current energy system. One way to use the solar radiation is photosynthesis. The photosynthesis evolved over 3.5 billion years ago in organisms and is known as the most efficient way to capture and use solar energy.

Hydrogen, which can be generated as a by-product of the metabolism in photosynthetic bacteria is considered the ideal alternative energy source for the future. Hydrogen, compared to fossil or atomic fuels, produces virtually no pollution. Also hydrogen which can be stored as both a gas and a liquid has a high potential to be a renewable substitute for fossil fuels.

The generation of hydrogen by photosynthetic bacteria is usually the result of proton (H+) fixation by the enzymes involved in nitrogen fixation, in which the energy required is entirely derived from light dependent reactions. Nitrogenase are enzymes used by some organism to fix atmospheric nitrogen gas and form a complex having two crucial components, i.e., NifH (dinitrogenase reductase) and NifD-NifK (dinitrogenase) requiring ATP (adenosine triphosphate) to function (Peters and Szilagyi 2006. Curr. Opin. Chem. Biol. 10: 101-108). The mechanism for generating hydrogen has not been completely elucidated and the molecular complexities of the nitrogenase and their sensitivity to oxygen have made the progress in the field of hydrogen generation even slower. Therefore, to the present, much of the work in the hydrogen production using photosynthetic bacteria has been focused on the optimization of light dependent reactions rather than studying the enzymes involved in the hydrogen or nitrogen generation. In other words, the research has been focused on the optimization of incubators for more light to be utilized as energy source by bacteria to generate hydrogen.

However, this approach has the limit in that it depends on the energy utilization efficacy unique to the light device used. Therefore, researches are required to direct for more energy to be used for the metabolism related to the generation of hydrogen and at the same time researches to develop methods for activating or stabilizing hydrogen producing enzymes are required.

Photosynthetic bacteria are classified into purple non-sulfur bacteria, purple sulfur bacteria, green non-sulfur bacteria, and green sulfur bacteria. The photosynthetic reaction by these bacteria is characterized by no oxygen production during the photosynthesis not like that of algae or plant.

Among them, purple non-sulfur bacteria belong to genus Rhodobacter are able to grow in a variety of metabolic condition such as aerobic, anaerobic light dependent or anaerobic light independent conditions. Their ability to convert solar energy to hydrogen makes Rhodobacter the major research subject in the development of alternative energies. During the last fifty years, gene manipulation methods have been well established in Rhodobacter, and nitrogenases and other components involved in hydrogen production are relatively well identified. For example Rhodobacter sphaeroides KCTC 12085 (Lee et al. 2002. Appl. Microbiol. Biotechnol. 60: 147-153) is a natural isolated strain having hydrogen producing capacity and high resistance to salts and can be genetically manipulated to produce variants having an improved hydrogen production.

However, further development/researches are required to use Rhodobacter in commercial scale due to its low efficiency in hydrogen production and the fact that the hydrogen generation is highly decreased in the presence of ammonia.

### SUMMARY OF THE INVENTION

The present disclosure is to provide methods for producing hydrogen using photosynthetic bacteria through stabilization and activation of enzymes involved in the hydrogen production, and to provide methods to improve the efficiency of hydrogen production.

In one aspect, the present disclosure provides a method to produce hydrogen comprising a step of culturing photosynthetic bacteria under a photosynthetic condition and in the presence of alcohol.

In one embodiment, the photosynthetic bacteria are a microorganism which belongs to genus Rhodobacter.

In other embodiment, the photosynthetic bacteria are selected from the group consisting of Rhodobacter sphaeroides, Rhodobacter capsulatus, R. apigmentum, R. azotoformans, R. blasticus, R. gluconicum, R. litoralis, R. massiliensis and R. veldkampii.

In still one embodiment, the alcohol is selected from the group consisting of methanol, ethanol, propanol, isopropanol and butanol.

In still one embodiment, the alcohol is ethanol.

In one embodiment, the alcohol is used to activate a nitrogenase.

In other embodiment, the present methods can be performed in the presence of ammonia.

In one embodiment of the present methods, the alcohol in the culture medium is not consumed.

In other embodiment, the alcohol is contained in the culture medium at the concentration of about from 0.05 vol % to 2 vol %.

In other embodiment, the alcohol is added at the beginning, for example, simultaneously with the inoculation of the bacteria, of the culturing step and/or at the early stage of the culturing step.

In still other embodiment, the photosynthetic bacteria is cultured at about 20° C. to 37° C., under an anaerobic or micro-aerobic atmosphere and with about 3-300 Watts/m² of light.

In other aspect the present disclosure provides methods for improving the efficiency of hydrogen production in the presence of alcohol under a photosynthetic condition.

In other aspect the present disclosure provides methods for producing hydrogen using organic waste such as food waste comprising a step of culturing photosynthetic bacteria under a photosynthetic condition and in the presence of alcohol.

The foregoing summary is illustrative only and is not intended to be in any way limiting. Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 shows the accumulative hydrogen production and growth curve of wild type strain R. sphaeroides under the photosynthetic condition. Sistrom minimal medium containing ammonium ion was used. Ethanol was used as a final concentration of 0.1 vol % and 0.5 vol % and the control did not contain any ethanol.

FIG. 2 shows the activity of nitrogenase of wild type strain R. sphaeroides under the photosynthetic condition. Ethanol was used as a final concentration of 0.2 vol % and the control did not contain ethanol. The activities were indicated by dividing the ethylene produced by the bacteria by the time and the number of cells.

FIG. 3 shows the amount of the accumulated hydrogen production by R. sphaeroides under the photosynthetic condition. R. sphaeroides wild type and NifDK mutant in which genes for hydrogen production enzymes were deleted were used. Ethanol was used as a final concentration of 0.5 vol % and the control did not contain ethanol. The time was indicated as a difference between the initial hydrogen production point and the hydrogen production measurement point.

FIG. 4 shows the accumulative hydrogen production and growth curve of wild type strain R. sphaeroides under the photosynthetic condition. The bacteria were grown in the presence of 0 mM or 2 mM ammonium ion, respectively and in the presence of 0.2 vol % ethanol. The control did not contain ethanol.

FIG. 5 shows the accumulative hydrogen production and growth curve of wild type strain R. sphaeroides under the photosynthetic condition. The ethanol (0.5 vol %) was added at the different cell growth stage of 10 KU, 100 KU and 300 KU. The control did not contain ethanol.

FIG. 6 shows the accumulative hydrogen production and growth curve of wild type strain R. sphaeroides under the photosynthetic condition. The Sistrom media containing ammonium ion and methanol, ethanol, propanol or butanol at the concentration of 0.2 vol %, 0.5 vol %, 0.5 vol % and 0.2 vol %, respectively were used.

### DETAILED DESCRIPTION OF THE EMBODIMENTS

The present disclosure has been based on the discovery that the alcohol could dramatically increase the ability of the photosynthetic bacteria to produce hydrogen.

In one aspect, the present disclosure relates to methods to produce hydrogen, which comprises a step of incubating the photosynthetic bacteria in the presence of alcohol under the photosynthetic condition.

In other aspect, the present disclosure relates to methods for improving the efficiency of hydrogen production in the presence of alcohol under the photosynthetic condition.

In accordance of the present disclosure the photosynthetic bacteria which may be used include, but are not limited to, Rhodobacter sphaeroides, Rhodobacter capsulatus, R. apigmentum, R. azotoformans, R. blasticus, R. gluconicum, R. litoralis, R. massiliensis and R. veldkampii. In one embodiment, R. sphaeroides or R. capsulatus are used. In still other embodiment, R. sphaeroides is used.

In accordance of the present disclosure, a photosynthetic condition or the condition under which the photosynthesis is to occur is the condition where the optimal amount of light, optimal temperature and/or optimal amount of air are present for the photosynthesis to occur. The skilled person in the art would be able to select and optimal condition according to the photosynthetic bacteria utilized. For example, the photosynthesis may be performed at about 20 to 37° C., anaerobic or micro-aerobic condition and about 3-300 Watts/m² of light. The micro-aerobic condition refers to less than about 5% of oxygen. In accordance of the present disclosure, the photosynthesis is performed for 120 hrs under the following condition: 3-300 Watts/m² of light, 30° C., anaerobic.

R. sphaeroides produces hydrogen gas using nitrogenase and thus the amount of hydrogen produced are determined by the degree of activity of the enzymes involved. However, the amount of hydrogen produced may be decreased depending on the activity of a hydrogenase containing Ni—Fe that uses the hydrogen produced (uptake-Hydrogenase, Appel et al. 2000. Arch Microbiol. 173: 333-338). It therefore can be said that the efficiency of R. sphaeroides is determined by the degree of relative activity of the two enzymes. In accordance of the present disclosure, the alcohols which may be used for the present disclosure are not particularly limited as long as it confer the present effects and include, but are not limited to, for example, methanol, ethanol, propanol, isopropanol or butanol. In one embodiment, the alcohols which may be used are ethanol, propanol or butanol. In other embodiment, the alcohols which may be used are butanol or ethanol.

The alcohols which may be used for the present disclosure may be added to the culture media up to the concentration that does not inhibit the growth of the bacteria, which for example, from about 0.05 vol % to about 2 vol % of the media used. In one embodiment, the alcohol may be added in the concentration from about 0.1 vol % to about 1 vol %.

The alcohol may be added at a proper time during the bacterial growth. For example the alcohol may be added simultaneously with the inoculation of the photosynthetic bacteria or at an initial stage of the bacterial growth. The initial stage means the stage when the cell concentration reaches about 1-150 KU.

The alcohol added activates the nitrogenase. In other aspect the present invention relates to methods for activating nitrogenase involved in the hydrogen production in photosynthetic bacteria.

The amount of alcohol used is negligible compared to that of hydrogen produced. The increased hydrogen production is due to the activation of nitrogenase by the alcohols not due to the use of the alcohol added as an energy source by the photosynthetic bacteria, for example, R. sphaeroides.

Therefore, in one embodiment of the present methods, the alcohols added are not consumed. Thus, in a continuous process of hydrogen production, no additional alcohols need to be added to keep the increased hydrogen production, resulting in increasing the efficiency of the hydrogen production.

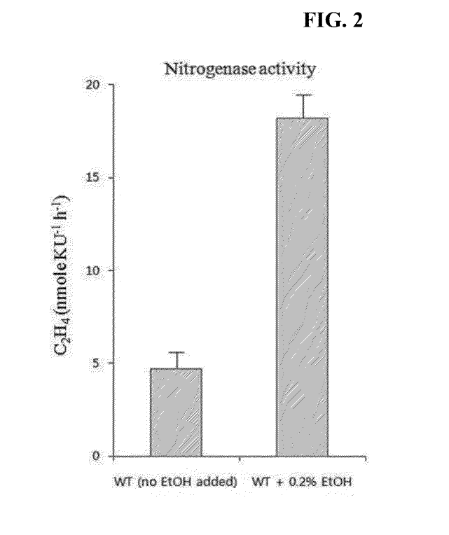

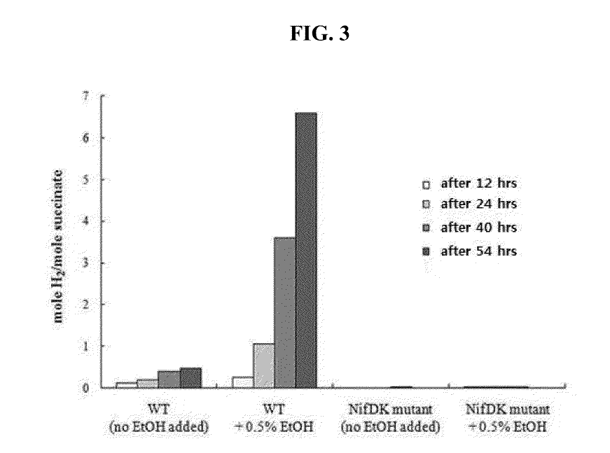

In one embodiment, it was measured that the activity of the nitrogenase was increased 4 times compared to the control after addition of ethanol (refer to FIG. 2), which did not observed in the mutant strain which does not express nitrogenase (FIG. 3). These results indicate that the increased hydrogen production is due the activation of nitrogenase by alcohols.

Therefore, the alcohol used in the present methods leads to the activation of nitrogenase and thus results in the increased production of hydrogen. But the theory is not limited thereto.

In other aspect, the present methods relates to the methods which is able to efficiently produce hydrogen in the presence of ammonium.

It is known that the inhibition of nitrogenase by the ammonium ion is one of the obstacle to overcome in the hydrogen production method using microorganism belong to the genus Rhodobacter. The hydrogen production by the present method is not sensitive to the presence of ammonium. It has been known that the expression and activity of nitrogenase are dramatically decreased by a variety of regulators in the presence of ammonium ion in media (Masepohl et al. 2002. J. Mol. Microbiol. Biotechnol. 4: 243-248). This is due to the fact that the nitrogen fixing reaction requires a large amount of ATP. The regulatory process of nitrogenase is largely composed of three levels: the highest level of regulation includes the regulation of a transcription factor NifA, which regulates the transcription of a gene for the nitrogenase nifHDK. NtrC is phosphorylated by NtrB in the absence of ammonium, and the phosphorylated NtrC increases the transcriptional level of nifA gene, and PII protein GlnB helps this process by associating with the NtrB. The second level of regulation includes the regulation of the activation of NifA, in which PII proteins such as GlnB and GlnK are involved. At this level of regulation the degree of transcriptional level of the nitrogenase is determined by regulating the activation of NifA. The third level of regulation regards to the regulation of the activity of the nitrogenase, in which the final activity of the nitrogen fixing enzyme is determined by DraT and DraG depending on the concentration of ammonium ion.

Particularly, the hydrogen production by photosynthetic bacteria, for example, R. sphaeroides, is carried out by the nitrogenase and when the nitrogen source is not present enough in the media, the nitrogenase synthesizes NH₄⁺ and at the same time, produces hydrogen gas. However, the nitrogenase activity is inhibited by the ammonium ion, which also leads to the inhibition of hydrogen production.

This results in the disadvantage of requiring additional cost and time to regulate the concentration of ammonium ion, particularly in hydrogen production using organic waste. The present methods however obviate this problem in which the hydrogen production is possible in the presence of ammonia. In one embodiment, this is hypothesized that it is possible by the suppression of the function of PII protein which regulates the activity of nitrogenase by recognition of the presence of ammonium ion.

The present disclosure is characterized by the use of alcohol to increase the production of hydrogen by photosynthetic bacteria by inducing the activation of the nitrogenase in the bacteria. Thus, any methods known in the art to produce hydrogen by photosynthetic bacteria may be employed for the present methods. The general methods to generate hydrogen gas using photosynthetic bacteria may be found in Lee et al. 2002. Appl. Microbiol. Biotechnol. 60: 147-153; Kim et al. 2006. Int. J. Hydrogen Energy 31: 121-127; or Korean Patent No. 0680624. The specific conditions under which the hydrogen is produced using photosynthetic bacteria in the presence of alcohol, for example, such as conditions for photosynthesis, culture condition and/or the amount of alcohol added can be easily determined by the skilled person in the art.

The present disclosure is further explained in more detail with reference to the following examples. These examples, however, should not be interpreted as limiting the scope of the present invention in any manner.

### EXAMPLES

### Example 1

### Hydrogen Production Using Photosynthetic Bacteria in the Presence of Alcohol

For the hydrogen production, R. sphaeroides 2.4.1(ATCC BAA-808, Cohen-Bazire et al. 1956. J. Cell. Comp. Physiol. 49: 25-68) or R. sphaeroides KCTC 12085 were used. To grow the cells, basically Sistrom minimal medium [20 mM KH₂PO₄, 3.8 mM (NH₄)₂SO₄, 34 mM succinate, 0.59 mM L-glutamate, 0.30 mM L-aspartate, 8.5 mM NaCl, 1.05 mM nitrilotriacetic acid, 1.2 mM MgCl₂6H₂O, 0.23 mM CaCl₂7H₂O, 25 M FeSO₄7H₂O, 0.16 M (NH₄)6Mo₇O₂₄4H₂O, 4.7 M EDTA, 38 M ZnSO₄7H₂O, 9.1 M MnSO₄H₂O, 1.6 M CuSO₄5H₂O, 0.85 M Co(NO₃)₂6H₂O (II), 1.8 M H₃BO₃, 8.1 M Nicotinic acid, 1.5 M Thiamine HCl, 41 nM biotin (Sistrom, W. R, 1962. J. Gen. Microbiol. 28: 607-616)] were used with the following modifications. To optimize the activity of nitrogenase, Ammonium molybdate was replaced with the same amount of Sodium molybdate, or 7 mM of L-glutamate was used instead of ammonium sulfate, or succinate was replaced with 30 mM malate. To investigate the effect of ammonium, 2 mM Ammonium chloride was added.

R. sphaeroides was inoculated at the concentration of 10⁸ CFU/ml in 10 ml of Sistrom minimal medium and incubated for 120 hrs. to test the hydrogen production at the following condition: 10 Watts/m², 30° C., anaerobic. To measure the hydrogen produced, the cells were incubated in a serum vial which was air tight. Before use the air in the vial was replaced with argon gas to remove oxygen. During the reaction, an aliquot of gas in the gas phase was sampled from the vial using the gas-tight syringe, which was further analyzed by a Gas Chromatography (GC, Shimadzu, Japan).

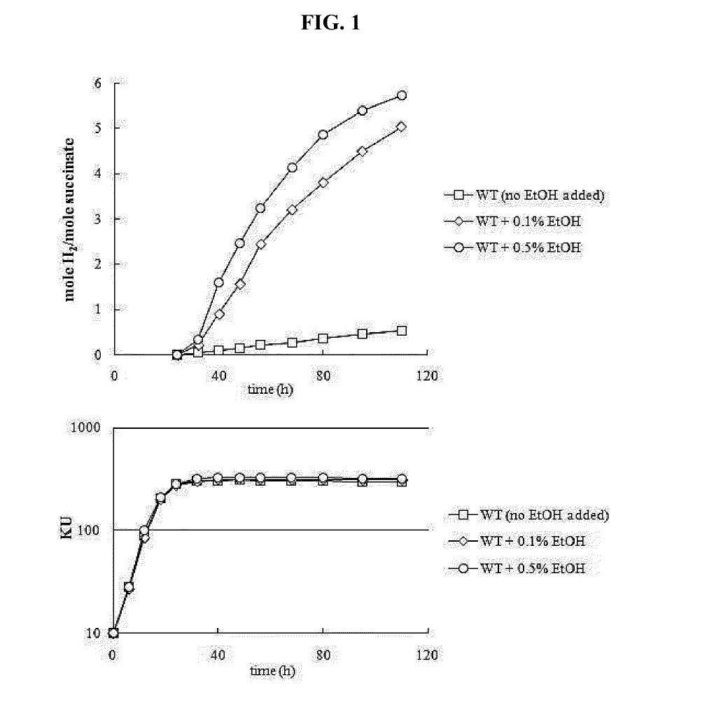

The results are shown in FIG. 1, which represents the accumulated amount of hydrogen produced and the growth curve. The Sistrom medium containing ammonium ion was used for the experiment and EtOH was used at the final concentration of 0.1 vol % and 0.5 vol %, and the control did not contain EtOH. As shown in FIG. 1, at each condition tested, the growth of bacteria was not affected. In the culture using the Sistrom medium, the hydrogen started to be generated when the growth was near the completion, the control cells which did not contain ethanol produced hydrogen at the efficiency of 0.52 mole H₂/mole succinate. In contrast, the bacteria containing ethanol 0.1 vol % or 0.5 vol %, each produced hydrogen at the efficiency of 5.04 mole H₂/mole succinate and 5.72 mole H₂/mole succinate, respectively, which is 10 times higher than the amount observed with the control.

### Example 2

### Measurement of Activity of Nitrogenase by the Addition of Ethanol

The activity of nitrogenase was measured by the rate of ethylene (C₂H₄) produced using the acetylene (C₂H₂) as a substrate. The nitrogenase has an ability to produce hydrogen and ethylene by reducing proton (H+) and acetylene in addition to using N₂ as substrate (Kern et al. 1992. Appl. Microbiol. Biotechnol. 37: 496-500). The activity of the nitrogenase was measured by incubating R. sphaeroides under the photosynthetic condition of 10 Watts/m² of light. 10 ml of the bacteria (10⁸ CFU/ml) was placed in a serum vial (65 ml) and incubated under the light until the KU (Klett Unit) reached 150 to 200. To prevent the de novo protein synthesis during the incubation period, chloramphenicol was used at 50 μg/ml and the air in the vial was replaced with argon before use. Acetylene was injected into the vial to occupy 10% of the entire air phase using a gas-tight syringe. The cells were first incubated for 10 min without light and the reaction started under the 10 Watts/m² light. The amount of ethylene produced was measured by withdrawing an aliquot of the gas in the gas phase using a gas-tight syringe and by subject them to GC analysis.

The results are shown in FIG. 2, which shows the activity of the nitrogenase of R. sphaeroides tested as above. The test sample contained 0.2 vol % of EtOH and the control did not. As shown in FIG. 2, the activity of nitrogenase of the test sample was found to be 18.2 nmole KU⁻¹ h⁻¹, which is 4 times higher than that of the control which had 4.7 nmole KU⁻¹ h⁻¹.

These results demonstrate that the hydrogen production by R. sphaeroides can be increased by the addition of alcohol and the enhancement is due to the increasing activity of the nitrogenase.

### Example 3

### Construction of Variant Having Nitrogenase Deletion and Testing the Effect of Ethanol on the Hydrogen Production

The chromosome of R. sphaeroides 2.4.1 was extracted according to the conventional methods and used as a template for PCR to amplify a 0.6 kb fragment of N-terminal region of NifD and a 0.7 kb fragment of C-terminal region of NifK, using the primers as follows: (NifD-Forward: 5′-CCG AGA CCA ACA TGA AGC-3′, NifD-Reverse: 5′-TCG CGA TAT GGT GGC-3′, NifK-Forward: 5′-TAC CGC ATG TAT GCG-3′, NifK-Reverse: 5′-CGA ACG AGA TGT CGG-3′). Then each of the amplified fragments was inserted into a T-vector called pMD20-T (Takara Bio, Japan) to construct pMD20-NifD and pMD20-NifK, respectively, the amplification fidelity of which was confirmed by sequencing analysis.

After that pMD20-NifD was digested with XbaI and SmaI to obtain a 0.6 kb fragment of NifD, and it was also digested with SmaI and PstI to obtain a 2.0 kb fragment containing streptomycin and spectinomycin resistance genes (Sm^{r}/Sp^{r}) and a transcription and a translation termination sequence. The fragments obtained then were ligated into a pBS (Stratagene) to obtain pBS-NifDS/S. Then the pBS-NifDS/S was digested with XbaI and PstI to obtain a 2.6 kb fragment. Also pMD20-NifK was digested with PstI and SacI to obtain a 0.7 kb fragment containing NifK C-terminal region. Then the 2.6 kb fragment and 0.7 kb fragment were cloned into a suicide vector pLO1 (Lenz O et al. 1994. J. Bacteriol. 176: 4385-4393) to obtain pLO-NifDK, which was then used to delete the NifD and NifK gene present on the chromosome. The E. coli cells containing the pLO-NifDK plasmid were selected using Kanamycin, Streptomycin and Spectinomycin at the concentration of 25, 50 and 50 μg/ml, respectively. The resulting plasmid was then transformed into E. coli S17-1 cells and transferred into R. sphaeroides by conjugation described as below. The E. coli cells containing the plasmid were mixed with R. sphaeroides and placed on an agar plate and allowed for the conjugation to occur for 6 to 12 hours. The cells were then spread on to a Sistrom/proline-limited agar plate containing Sm and Sp and Km to select the successfully conjugated cells.

E. coli S17-1 is an auxotroph that requires proline for growth and thus cannot grow in a plate depleted with proline. The non-transformants were removed by growing the cells in the presence of 25 μg/ml of Kanamycin.

Also pLO1 vector is a suicide vector and cannot be amplified in R. sphaeroides and thus a single crossover was induced through a homologous recombination on the R. sphaeroides 2.4.1 chromosomal DNA.

In addition pLO1 vector contains a sacB gene encoding Levansucrase, which kills the cell by forming a polymer of sucrose in the presence of sucrose.

Therefore, by using the streptomycin and spectinomycin each at 50 μg/ml and sucrose at 15% by weight, the colonies in which a double crossover was occurred can be selected, which were sensitive to Kanamycin but resistant to Streptomycin and Spectinomycin. These colonies did not express NifD and NifK, and thus did not have the nitrogenase activity in the cells.

The wild type R. sphaeroides and the nitrogenase deficient strain (NifDK mutant) as constructed above were used for the production of hydrogen under the same condition as described in Example 1 using 10 Watts/m² light. The Sistrom medium containing ammonium ion was used and the growth analysis, the hydrogen production, and the analysis of the hydrogen produced were performed as described in Example 1.

The results are shown in FIG. 3, which shows the accumulated amount of hydrogen produced by R. sphaeroides. As shown in FIG. 3, no difference in the growth was observed between the two strains. The wild type strain showed the increased hydrogen production by the addition of EtOH. In contrast, the hydrogen production was not observed regardless of the presence of ethanol in the mutant strain. The results demonstrate that the enhancement of the hydrogen production observed was due to the activation of the nitrogenase by the ethanol added to the cells.

### Example 4

### The Effect of Ethanol on the Enhancement of Hydrogen Production in the Presence or the Absence of Ammonium Ion

It was tested whether the ammonium ion inhibits the activity of nitrogenase in the presence of ethanol.

The wild type R. sphaeroides cells were grown under 10 Watts/m² of light as described in Example 1 and tested for the production of hydrogen. To test of the effect of ammonium ion, a modified medium optimized for the hydrogen production which did not contain or contained 2 mM ammonium chloride (Lee et al. 2002. Appl. Microbiol. Biotechnol. 60: 147-153) was used for the experiment.

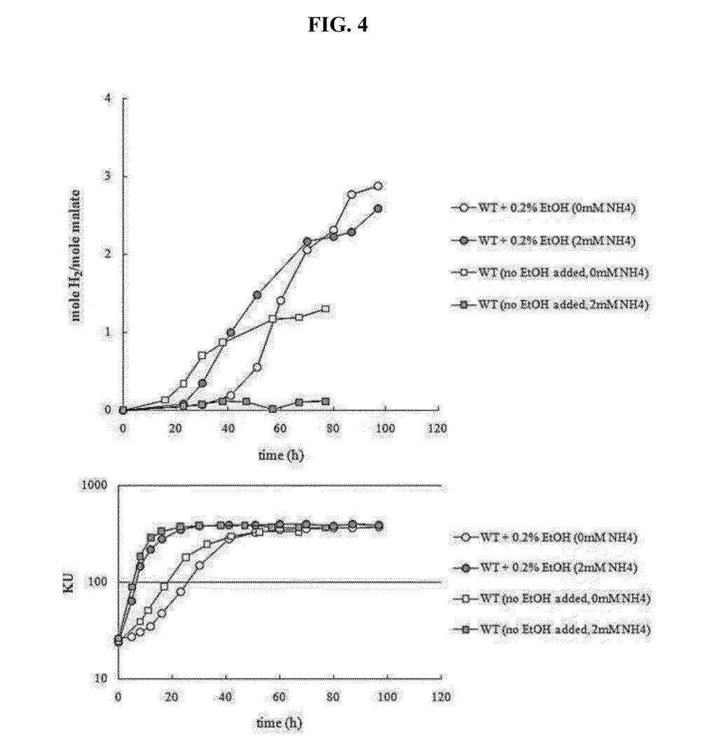

The hydrogen produced was analyzed as described in Example 1. The results are shown in FIG. 4, which shows that the growth rate became slower in the cells grown in a medium containing 0 mM ammonium chloride compared to the cells in 2 mM ammonium ion regardless of the presence of ethanol. However, the number of cells was found to be similar in all the conditions tested.

In the modified medium used as described above, the hydrogen gas is produced from the initial stage of the growth. It was measured that in the presence of 2 mM ammonium ion, the hydrogen gas producing ability (activity) was remained not less than 80% compared to that in the presence of 0 mM ammonium ion, in the presence of 0.2% ethanol (FIG. 4). In contrast, the hydrogen production by the control cells which did not contain any ethanol showed the decrease under 10% in the presence of the ammonium ion. This demonstrates that the increased resistance to the ammonium ion by the addition of ethanol showing that more than 80% of the nitrogenase activity was remained after the addition of ethanol in the presence of ammonium ion.

### Example 5

### The Effect of Ethanol on the Hydrogen Production in Other Purple Non-Sulfur Bacteria

Other purple non-sulfur bacteria, Rhodobacter capsulatus and Rhodospirillum rubrum, which are widely used for testing hydrogen production, were used to test the effect of alcohol on the hydrogen production.

Rhodobacter capsulatus SB1003 and Rhodospirillum rubrum UR1 selected for the experiment due to their well characterization. For the growth of R. capsulatus, a modified Sistrom medium depleted with ammonium ion was used; and for the growth of R. rubrum, MG minimal medium was used as described in Lehman and Roberts. 1991. J. Bacteriol. 173: 5705-5711.

Each strain was inoculated to the medium as described above at 10⁸ CFU/ml and incubated for 120 hours under the following condition: 10 Watts/m² of light, 30° C., and anaerobic condition and the amount of hydrogen produced was tested. The ethanol was added at 0.2 vol % and the cell growth, and hydrogen production and analysis were performed as described in Example 1.

The results are shown in Table 1 below. As shown in table 1, the hydrogen production was increased 1.5 times in R. capsulatus by the addition of ethanol, but the increasing effect was not observed in R. rubrum.

TABLE 1
The effects of ethanol on the hydrogen production in various purple
non-sulfur bacteria

| | Name of the strain used | | |
|---|---|---|---|
| | R. sphaeroides | R. capsulatus | R. rubrum |
| fold increase | 2.21 | 1.53 | 0.89 |

(Fold calculation: amount of hydrogen produced in the presence of alcohol/amount of hydrogen produced in the absence of alcohol)

### Example 6

### Determination of the Optimal Time and Concentration of the Alcohol Added to Increase the Hydrogen Production

To determine the optimal concentration and time of the alcohol added, the accumulated amount of hydrogen produced was measured in R. sphaeroides in the presence of varying amounts of ethanol from 0.01 vol % to 2 vol % (volume/volume %) by incubating them under the same condition as described in Example 1. It was observed that 2% ethanol inhibited the growth of cells and also the hydrogen produced was very much decreased. Also, ethanol less than 0.05% was not shown to increase the hydrogen production. Therefore it was determined that optimal concentration was found to be from 0.05 vol % to 2 vol %, particularly 0.1 vol % to 1 vol %.

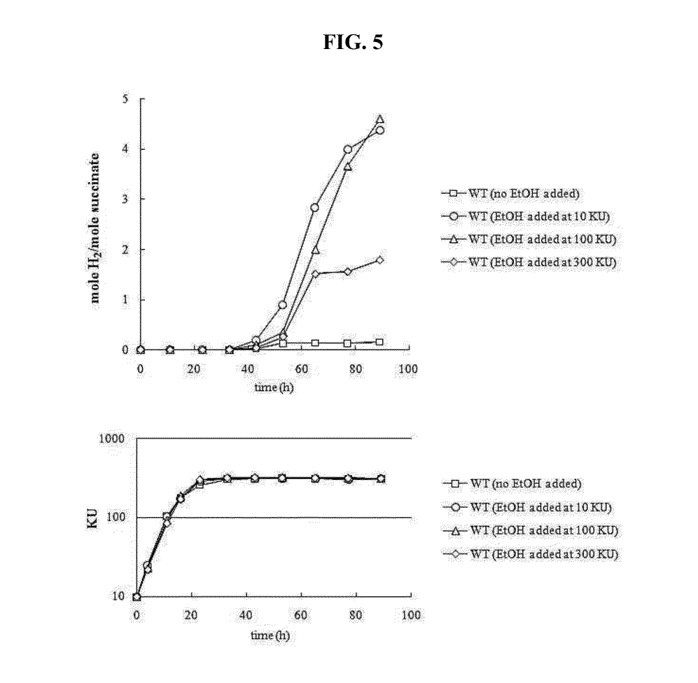

FIG. 5 shows the accumulated amount of hydrogen produced and the growth curve in which alcohol was added at different time points. The cells that were inoculated to be 10 KU became near 300 KU after 24 hrs and there was no increase observed. However, the hydrogen production was observed at 30 hrs after the inoculation while there was no increase in cell number. For this reason, the ethanol was added at different time points.

As shown in FIG. 5, it was found that the ethanol added simultaneously with the cell inoculation or when the cells reached 100 KU at the early growth stage showed a higher increasing effect on the hydrogen production compared to the addition at 300 KU at the later stage of the growth. This indicates that the presence of ethanol at the early stage of the growth is more beneficial for the hydrogen production even though the actual hydrogen production was occurred 30 hrs after the inoculation, which demonstrates that the ethanol was functioned most effectively during the vigorous growing stage of the cells.

### Example 7

### The Change of Concentration of Succinate and Ethanol in the Hydrogen Producing Medium

The concentration of succinate, a major carbon sources in a medium, ethanol, and that of ammonium ion that affects the activity of nitrogenase were measured. The hydrogen production by cells was performed as described in Example 1. The amount of succinate was measured using HPLC. For HPLC, Aminex HPX-87H organic acid column (Bio-Rad, USA) was used and filtered 30 μl of medium was injected thereto. As a mobile phase 0.01 M H₂SO₄ was used, the oven temperature used was 60° C., and the rate of the mobile phase was set to 0.6 ml/min. The concentration of ethanol in the medium was by Enzychrom ethanol assay kit (Bioassay Systems, USA) using alcohol dehydrogenase. In the presence of ethanol, the rate of consumption of succinate in the presence of alcohol was not different from that in the absence of alcohol, the succinate was observed to be consumed continuously during the cell growth (Table 2). In contrast, it was found that the ethanol concentration remained to be constant at 5 μl/ml (0.5%) which was the concentration at the time of addition (Table 3). This results indicate that not like succinate which was used as a carbon source, the ethanol added was not used as a carbon source and remained constant, affecting the activity of nitrogenase.

TABLE 2
The concentration of succinate during R. sphaeroides growth under
photosynthetic condition.

Succinate(mM)
Ethanol added to the medium

| Time(hours) | − | + |
|---|---|---|
| 0 | 36.98 ± 2.58 | 33.92 ± 1.08 |
| 36 | 35.41 | 31.24 |
| 49 | 24.80 | 23.03 |
| 130 | 9.72 | 4.81 |
| 197 | 3.19 | [symbol] |

[symbol]

indicates data missing or illegible when filed

TABLE 3
The concentration of ethanol during R. sphaeroides growth under
photosynthetic condition.

Ethanol(%)

| Time(hours) | − | + |
|---|---|---|
| 0 | ND* | 0.62 ± 0.01 |
| 36 | ND | 0.63 ± 0.02 |
| 49 | ND | 0.56 ± 0.01 |
| 130.5 | ND | 0.65 ± 0.01 |
| 197 | ND | 0.53 ± 0.0 |

### Example 8

### Effects of Various Alcohols on the Production of Hydrogen

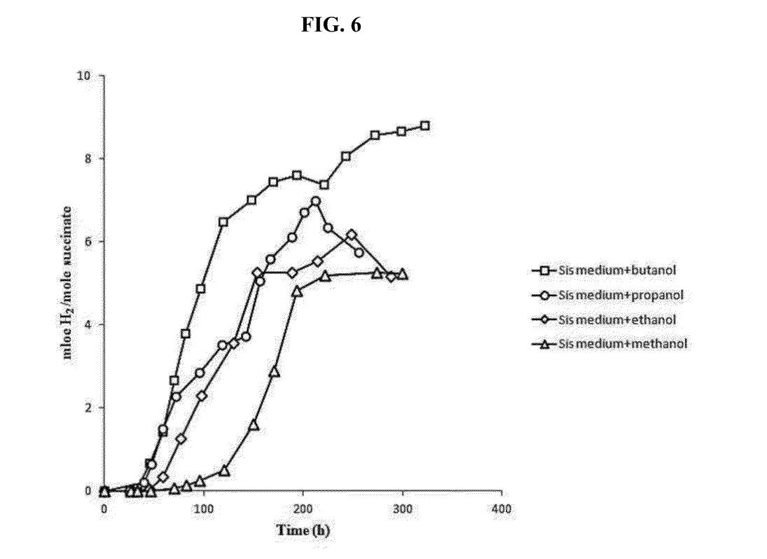

In addition to ethanol, other alcohols, that is, methanol (CH₃OH), propanol (CH₃CH₂CH₂OH), isopropanol [(CH₃)₂CHOH], butanol [CH₃(CH₂)₂CH₂OH], isoamyl alcohol, [(CH₃)₂CHCH₂CH₂OH] were tested. Under the same condition as used in Example 1, each alcohol was added to the cells at the concentration of 0.1%, 0.2%, 0.5%, or 1.0%. Results are shown in FIG. 6. Methanol was shown to inhibit the cell growth at 1.0%, and others have no effect on the cell growth. With regard to the hydrogen production, at the 0.1%, 0.2%, and 0.5% concentration the hydrogen production was increased with 0.2% being the highest effect. For the propanol, at all the concentration tested, i.e., 0.1%, 0.2%, 0.5%, 1.0%, the hydrogen production was increased with 0.5% being the highest effect. The isopropanol was shown to increase by about 35% of hydrogen production, which was not comparable to the effect conferred by ethanol, propanol, and butanol. In the case of butanol, at the 0.1%, 0.2%, and 0.5% concentration, the hydrogen production was increased with 0.2% being the highest effect. In the case of isoamyl alcohol, it was found to decrease the hydrogen production.

With respect to the use of substantially any plural and/or singular terms herein, those having skill in the art can translate from the plural to the singular and/or from the singular to the plural as is appropriate to the context and/or application.

The various singular/plural permutations may be expressly set forth herein for sake of clarity. Although a few embodiments of the present disclosure have been shown and described, it would be appreciated by those skilled in the art that changes may be made in this embodiment without departing from the principles and sprit of the invention, the scope of which is defined in the claims and their equivalents.

What is claimed is:

1. A method of producing hydrogen comprising a step of culturing photosynthetic bacteria in a medium under a photosynthetic condition and in the presence of an alcohol.

2. The method of claim 1, wherein the photosynthetic bacteria is Rhodobacter sp.

3. The method of claim 1, wherein the photosynthetic bacteria is selected from the group consisting of Rhodobacter sphaeroides, R. capsulatus, R. apigmentum, R. azotoformans, R. blasticus, R. gluconicum, R. litoralis, R. massiliensis and R. veldkampii.

4. The method of claim 1, wherein the alcohol is one or more selected from the group consisting of methanol, ethanol, propanol, isopropanol and butanol.

5. The method of claim 1, wherein the alcohol affects the activity of a nitrogenase.

6. The method of claim 1, wherein the method can be performed in the presence of ammonium.

7. The method of claim 1, wherein the alcohol is not consumed during the hydrogen production.

8. The method of claim 1, wherein the alcohol is included in the medium at a concentration from about 0.05 vol % to about 2 vol %.

9. The method of claim 1, wherein the alcohol is added to the medium at simultaneously with the inoculation of the photosynthetic bacteria, and/or at the early stage of the culturing step.

10. The method of claim 1, wherein the photosynthesis is performed at about 20° C. to 37° C., under an anaerobic or micro-aerobic atmosphere and about 3-300 Watts/m² of light.
